(19) United States
(12) Patent Application Publication
(10) Pub. No.: US 2019/0142569 A1
(43) Pub. Date: May 16, 2019

(54) Artificial Trachea and Method for Producing the Same

(71) Applicant: Kyoto University, Kyoto-shi (JP)

(72) Inventors: Tatsuo Nakamura, Kyoto-shi (JP); Yuji Inada, Kyoto-shi (JP); Keiji Shigeno, Kyoto-shi (JP)

(73) Assignee: Kyoto University, Kyoto-shi (JP)

(21) Appl. No.: 16/189,138

(22) Filed: Nov. 13, 2018

(30) Foreign Application Priority Data
Nov. 15, 2017 (JP) 2017-220048

(51) Int. Cl. A61F 2/04; A61L 27/24; A61L 27/54; A61L 27/56; A61L 27/40

(52) U.S. Cl. CPC A61F 2/04; A61L 27/24; A61F 2002/046; A61L 27/56; A61L 27/40; A61L 27/54

(57) ABSTRACT

Disclosed is an artificial trachea comprising: a hollow tubular base material whose cross-section in a direction orthogonal to the central axis of the tube has an approximately circular shape.

### CROSS-REFERENCE TO RELATED APPLICATION

This application claims benefit under Paris Convention of Japanese Patent Application No. 2017-220048 filed on Nov. 15, 2017, incorporated herein by reference in its entirety.

### BACKGROUND OF THE INVENTION

### Field of the Invention

The present invention relates to an artificial trachea and a method for producing the same.

### Description of the Related Art

In recent years, there have been increasing cases where reconstruction of a trachea and its bifurcation is needed, and there has been made a study on an artificially manufactured trachea (hereinafter also referred to as “artificial trachea”) that is used for reconstruction of the trachea and its bifurcation.

The artificial trachea is required to have properties that enable adequate supporting property to the lumen as well as rapid and reliable incorporation in a living body with little inflammatory reaction in the body tissues.

PCT International Publication WO01/024731 discloses an artificial trachea comprising a polypropylene mesh tube as a base material, around the outer periphery of which a polypropylene filamentous stent is wound in a spiral shape (or helically), an amorphous collagen thin layer on the surface of the base material, and fine fibrous collagen layers formed on the inner and outer surfaces of the amorphous collagen thin layer, the fibrous collagen layers being subjected to thermal crosslinking (see WO01/024731: claim 1, line 27 on page 3 to line 2 on page 4, FIG. 1).

JP 6-17715 U (utility model) discloses an artificial trachea comprising a polypropylene mesh tube as a base material, around the outer periphery of which a polypropylene monofilament is wound in a spiral shape, an amorphous collagen thin layer on the surface of the base material, and fine fibrous collagen layers on the inner and outer surfaces of the amorphous collagen thin layer, the fibrous collagen layers being subjected to thermal crosslinking (see JP 6-17715 U: paragraphs [0007] to [0008]).

The artificial trachea is inserted into a living body, and faces the outside at the inner surface of the airway. Therefore, the body makes an attempt to reject the artificial trachea out of the body as a foreign body (or foreign object). Thus, the artificial trachea is required to have higher biocompatibility and to be easily incorporated into the living body. To do this, the artificial trachea is first required to be scarcely recognized as a foreign body inside the living body, and thus it is preferred to have a structure as simple and uncomplicated as possible.

Meanwhile, the artificial trachea is used inside a living body over a long period of time, and is therefore required to have a frame part acting as a mechanical structure that is stable inside the living body over a longer period of time, and to have a mechanical strength capable of retaining the lumen of the artificial trachea over a longer period of time, even though it has a simple and uncomplicated structure.

The artificial trachea disclosed in WO01/024731 has an excellent biocompatibility and a mechanical strength, and the stent is further placed on the outer periphery of the tubular mesh base material. Therefore, the artificial trachea has a tubular double structure. The artificial trachea is entirely thickened by the stent as a support material, thus making it possible to give an artificial trachea having a larger size.

Therefore, when the artificial trachea disclosed in WO01/024731 is further used over a longer period of time, there is a higher possibility of giving mechanical stress to the living body. In today's increasing human life span, it is important that the artificial trachea can be used over a longer period of time. Since surgery performed on an older age person is associated with a higher risk on hid life, it is important that the artificial trachea can be used over a longer period of time.

With regard to the artificial trachea disclosed in JP 6-17715 U, the support material (or reinforcing material) of one monofilament is wound in a spiral shape on the outer periphery of the tubular base material. Comparing to the artificial trachea disclosed in WO01/024731, this artificial trachea is considered to be simpler and gives a smaller mechanical stress based on the support material. The artificial trachea of JP 6-17715 U also has a mechanical strength capable retaining the lumen therein.

However, the only single monofilament is used spirally as the support material for the entire tubular base material. Therefore, if this single monofilament is broken, the following problems can arise: the strength of the artificial trachea can totally decrease, and balance of the strength can be deteriorated.

Furthermore, the tubular base material, around the outer periphery of which the support material is wound spirally, may have drawbacks or cause problems as follows: The tubular base material lacks in extensibility in the longitudinal axis direction, and when a twisting force is applied in a spiral direction, the tubular base material is deformed into a collapsed shape, and thus the lumen is likely to be developed in a constriction form. To the contrary, when a twisting force is applied in a direction opposite to the spiral direction, the spiral support material is likely to be peeled from the tubular base material, etc.

In JP 6-17715 U, there is also a problem that it is not easy to treat (or deal) an artificial trachea having a complicated form such as a branching form or a form having a non-constant diameter of the trachea.

### SUMMARY OF THE INVENTION

The present inventors have intensively studied and found that an artificial trachea having a simple and uncomplicated structure can be obtained by replacing the form of the support material of the monofilament with another specific form. They have also found that such an artificial trachea is stable over a longer period of time inside a living body, and has a mechanical strength capable of retaining the lumen of the artificial trachea over a longer period of time, even though it has a simple and uncomplicated structure, thus completing the present invention.

Thus, the present invention provides, as one aspect, a novel artificial trachea, which comprises:

a hollow tubular base material whose cross-section in a direction orthogonal to the central axis of the tube has an approximately circular shape, the hollow tubular base material having meshes (or holes) on the side of the tube;

a plurality of annular support materials that are placed apart from each other on the outer periphery of the tubular base material; and

porous collagen layers on both the outside and the inside of the tubular base material, respectively, wherein

the tubular base material is made of polyolefin,

the annular support materials are made of at least one selected from polyolefin and polyamide, and

the tubular base material and the annular support materials are bonded to each other.

The present invention provides, in another aspect, a method for producing an artificial trachea, which comprises:

forming a mesh made of polyolefin (or a polyolefin mesh) in a hollow tubular shape to prepare a hollow tubular base material whose cross-section in a direction orthogonal to the central axis of the tube has an approximately circular shape, the hollow tubular base material having meshes (or holes) on the side of the tube;

placing a plurality of annular support materials apart from each other on the outer periphery of the tubular base material using a monofilament made of at least one selected from polyolefin and polyamide;

bonding the annular support materials to the tubular base material; and

placing porous collagen layers on both the outside and the inside of the tubular base material, respectively.

The artificial trachea according to an embodiment of the present invention has a simple and uncomplicated structure which comprises: a hollow tubular base material whose cross-section in a direction orthogonal to the central axis of the tube has an approximately circular shape, the hollow tubular base material having meshes on the side of the tube; a plurality of annular support materials that are placed apart from each other on the outer periphery of the tubular base material; and porous collagen layers on both the outside and the inside of the tubular base material, respectively, wherein the tubular base material is made of polyolefin, the annular support materials are made of at least one selected from polyolefin and polyamide, and the tubular base material and the annular support materials are bonded to each other.

Therefore, the artificial trachea according to the embodiment of the present invention has a higher biocompatibility and is more easily incorporated into a living body.

Meanwhile, the artificial trachea according to the embodiment of the present invention is stable over a longer period of time inside the living body, and also has a mechanical strength capable of retaining the lumen of the artificial trachea over a longer period of time, and can maintain stable performance over a longer period of time, even though it has a simple and uncomplicated structure.

### BRIEF DESCRIPTION OF THE DRAWINGS

FIG. 1 schematically shows a frame (backbone) (10) comprising annular support materials (4) and a tubular base material (2) having the annular support materials on the outer periphery thereof. FIG. 1(A) schematically displays the frame (10) viewed from the inside of the tubular base material (2) cut in the axial direction. FIG. 1(B) schematically exhibits the whole frame (10).

FIG. 2 schematically shows an artificial trachea (20) according to an embodiment of the present invention, having collagen layers (12, 14) on both the outside and the inside of the side of the frame (10). FIG. 2(A) schematically displays the artificial trachea (20) viewed from the inside thereof after being cut in the axial direction, and FIG. 2(B) schematically exhibits the whole artificial trachea (20).

FIG. 3 shows a photograph of a frame (backbone) of Example 1 comprising annular support materials and a tubular base material having the annular support materials on the outer periphery thereof.

FIG. 4 shows a photograph in which the outside of the frame in FIG. 3 is enlarged.

FIG. 5 shows a photograph in which the inside of the frame in FIG. 3 is enlarged.

FIG. 6 schematically depicts a mold.

FIG. 7 shows a photograph of an artificial trachea of Example 1.

FIG. 8 exhibits a scanning electron micrograph (at about 45 times magnification) of the outside collagen layer of the artificial trachea of Example 1.

FIG. 9 shows a photograph of a frame of Example 3. The frame of Example 3 has a branch, and has a Y-shaped form as a whole.

### DETAILED DESCRIPTION OF THE EMBODIMENT

Embodiments according to the present invention will be described in detail below with reference to the accompanying drawings. However, excessively detailed description may be omitted. For example, detailed description of already well-known matters and redundant description on substantially the same configuration may be omitted. This is intended to avoid the unnecessary redundancy of the following descriptions and to facilitate understandings by those skilled in the art.

It should be interpreted that the following descriptions are provided to enable those skilled in the art to fully understand the present invention, and are not intended to limit the claimed subject matter by those descriptions.

An artificial trachea according to an embodiment of the present invention comprises:

a hollow tubular base material whose cross-section in a direction orthogonal to the central axis of the tube has an approximately circular shape, the hollow tubular base material having meshes (or holes) on the side of the tube;

a plurality of annular support materials that are placed apart from each other on the outer periphery of the tubular base material; and

porous collagen layers on both the outside and the inside of the tubular base material, respectively, wherein

the tubular base material is made of polyolefin,

the annular support materials are made of at least one selected from polyolefin and polyamide, and

the tubular base material and the annular support materials are bonded to each other.

The artificial trachea according to the embodiment of the present invention includes a hollow tubular base material whose cross-section in a direction orthogonal to the central axis of the tube has an approximately circular shape, the hollow tubular base material having meshes (or holes) on the side of the tube.

As used herein, the “tubular base material” is not particularly limited as long as its cross-section in a direction orthogonal to the central axis of the tube has an approximately circular shape, and it has meshes on (or as) the side of the tube and is hollow, thus making it possible to give the objective artificial trachea.

The size of the meshes (or holes) which the tubular base material has on the side is that of the meshes through which general cells can invade, and is preferably the size capable of maintaining the mechanical strength, and can be appropriately selected.

The size of the tubular base material can be appropriately selected according to the location where the artificial trachea is actually used. Therefore, the diameter and the length of the tube can be appropriately selected.

The diameter of the tube may be entirely the same (uniform or constant) or non-constant (or changed). For example, the diameter may decrease from one end to the other end, and contrary the diameter may increase from one end to the other end.

The tubular base material may be linear or curved.

The tubular base material may have a tubular branch and may be, for example, Y-shaped. In other words, the tubular base material may have three or more ends. All three ends of the Y-shaped tubular base material may have the same diameter, two ends may have the same diameter and the other one may have different diameter, or all three ends have different diameters.

The tubular base material is made of polyolefin. The polyolefin substantially has no biodegradability and is stable inside a living body, but preferably has biocompatibility. The polyolefin is preferably polyethylene, polypropylene or ethylene-propylene copolymer. A net (mesh), a woven fabric, a knitting and the like can be formed of the polyolefin, and thus the tubular base material can be obtained from the mesh or the like. A thick fiber may be used as a fiber of polyolefin forming the mesh or the like. Alternatively, thin fibers may be used in combination, if necessary, for example, in the form of a braid or the like.

It is possible to use, as a polyolefin mesh or the like, commercially available products.

The polyolefin mesh includes, for example, a polypropylene mesh (BIRD (registered trademark) Mesh (trade name), manufactured by DAVOL INC.) and the like.

The artificial trachea according to the embodiment of the present invention includes a plurality of annular support materials that are placed apart from each other on the outer periphery of the tubular base material.

As used herein, the “annular support materials” are not particularly limited as long as they have an approximately circular shape and a plurality of annular support materials are placed apart from each other on the outer periphery of the tubular base material, thus making it possible to obtain the objective artificial trachea.

As used herein, the part, which retains (or maintains) a mechanical strength of the artificial trachea and comprises a tubular base material in which a plurality of annular support materials are placed on the outer periphery (for example, a combination of a tubular base material with an annular support material), is referred to as a “frame (or backbone)”.

The thickness and the shape of the annular support material in a direction perpendicular to the longitudinal axis (or transverse direction) are not particularly limited as long as the objective artificial trachea can be obtained.

The annular support material can be made of at least one selected from polyolefin and polyamide.

The polyolefin has substantially no biodegradability and is stable in a living body, but preferably has a biocompatibility. The polyolefin is preferably polyethylene, polypropylene or ethylene-propylene copolymer.

The polyamide has substantially no biodegradability and is stable in a living body, but preferably has a biocompatibility. The polyamide is preferably 6,6-nylon (registered trademark) or 6-nylon (registered trademark).

The annular support material can be formed of at least one selected from the polyolefin and the polyamide.

A plurality of annular support materials are placed apart from each other on the outer periphery of the tubular base material.

As used herein, “apart from each other” means that a plurality of annular support materials are apart from each other, and there may be exist a portion where the annular support materials are not apart from each other as a result of partial contact.

The interval, at which the annular support materials are placed apart from each other, can be appropriately selected as long as it is possible to ensure a mechanical strength to retain the lumen of the tubular base material.

The annular support materials are preferably placed in an equivalent interval.

As used herein, “equivalent interval” may be required to be visually recognized as approximately equal (or even) interval and does not mean strict equal interval. It is preferred from the viewpoint of balance of the strength of the tubular base material.

At least two annular support materials are placed, and it is preferred that at least three annular support materials are placed.

It is preferred that a plurality of annular support materials are placed such that the joint portions of three continuous annular support materials are not linearly aligned. This is preferred because the joint portions of the annular support materials are portions having a comparatively small strength (or comparatively weaker) with regard to the annular support materials, and thus when one annular support material comes off, it is possible to reduce influence on the other neighboring annular support materials.

In the artificial trachea according to the embodiment of the present invention, the tubular base material and the annular support materials are bonded to each other.

As used herein, the “bonding” means that the tubular base material and the annular support materials are bounded (or attached) to each other, and the bonding method, the bonding manner and the like are not particularly limited as long as the objective artificial trachea can be obtained.

The bonding method includes, for example, heat-bonding, suturing (sewing or fixing) with a filament suture, use of an adhesive, sewing into a net (or mesh) of the tubular base material and the like, and it is preferred to use heat-bonding and/or suturing with a filament suture.

The “heat-bonding” means a method for fusing a tubular base material, an annular support material or both of them with heating, and the heating means, the heating temperature and the like can be appropriately selected.

The “suturing with a filament suture” means sewing an annular support material on a tubular base material using a filament suture. The sewing method or the like can be appropriately selected. It is possible to use, as the filament suture, a suture which can be made of the same material as that of the above-mentioned annular support material (which can be made of at least one selected from polyolefin and polyamide).

Commercially available sutures can be used as the filament suture. The filament suture includes, for example, polypropylene filament suture (Prolene (trade name), manufactured by ETHICON Inc.), polypropylene filament suture (Surgipro (trade name), manufactured by COVIDIEN), polypropylene filament suture (suture with E-type needle (trade name), manufactured by Bear Medic Corporation) and the like.

It is more preferred to use both heat-bonding and suturing with a filament suture to increase the bonding strength.

The artificial trachea according to the embodiment of the present invention comprises porous collagen layers on both the outside and the inside of the tubular base material.

As used herein, the “porous collagen layer(s)” is/are not particularly limited as long as it is a layer(s) which can be made of collagen, and the collagen is porous, and the objective artificial trachea can be obtained.

The porous collagen includes a lot of pores (or voids), and the size of the pores may be a size through which general cells can invade.

The porous collagen preferably includes at least one selected from sponge collagen, thin film multilocular collagen and fine fiber collagen, and more preferably thin film multilocular collagen.

The thickness of the porous collagen layer can be appropriately selected.

The “artificial trachea” according to an embodiment of the present invention can have various forms based on the form of the tubular base material to be used.

For example, when the tubular base material has a linear form, the artificial trachea can have a linear form. When the tubular base material has a curved form, the artificial trachea can also have a curved form.

For example, when the tubular base material has a form having a constant diameter, the artificial trachea can also have a form having a constant diameter. When tubular base material has a form having a non-constant (or variable) diameter, the artificial trachea can also have a form having a non-constant diameter.

For example, when the tubular base material has a non-branching form, the artificial trachea can also have a non-branching form. When the tubular base material has a branching form, the artificial trachea can also have a branching form.

The above-mentioned forms of the artificial trachea can be appropriately used in combination.

Therefore, the artificial trachea according to an embodiment of the present invention can have at least one form selected from a linear form, a curved form, a form having a constant diameter, a form having a non-constant diameter, a non-branching form and a branching form. These forms can be appropriately used in combination.

The present invention provides a method for producing an artificial trachea according to an embodiment of the present invention. The production method comprises:

forming a mesh made of polyolefin (or a polyolefin mesh) in a hollow tubular shape to prepare a hollow tubular base material whose cross-section in a direction orthogonal to the central axis of the tube has an approximately circular shape, the hollow tubular base material having meshes on the side of the tube;

placing a plurality of annular support materials apart from each other on the outer periphery of the tubular base material using a monofilament made of at least one selected from polyolefin and polyamide;

bonding the annular support materials to the tubular base material; and

placing porous collagen layers on both the outside and the inside of the tubular base material, respectively.

The descriptions such as the “tubular base material”, the “polyolefin”, the “polyamide”, the “annular support material”, the “bonding”, and the “collagen layer”, which are mentioned for the artificial trachea according to the embodiments of the present invention, can be applied to the production method according to the embodiment of the present invention.

In the production method of the embodiment of the present invention, the method for placing a plurality of annular support materials apart from each other is not particularly limited as long as the annular support materials can be placed apart from each other on the outer periphery of the tubular base material using a monofilament made of at least one selected from polyolefin and polyamide, and the objective artificial trachea of the present invention can be obtained. The annular support materials may be placed after they are formed or while being formed, and the timing when they are formed is not limited. If necessary, one or more monofilament may be used.

In the production method of the embodiment of the present invention, the method for placing porous collagen layers is not particularly limited as long as the layers of porous collagen can be formed on both the outside and the inside of the tubular base material which has annular support materials on the outer periphery, and the objective artificial trachea of the present invention can be obtained.

The “placing porous collagen layers on both the outside and the inside of the tubular base material” preferably comprises immersing the tubular base material with annular support materials on the outer periphery into a dilute hydrochloric acid solution containing collagen, freezing the dilute hydrochloric acid solution containing collagen, and freeze-drying.

To make collagen to be placed easily on the tubular base material (or frame) including annular support materials on the outer periphery, an additional treatment(s) can be applied to the frame surface. The treatment includes, for example, a plasma treatment using an air plasma device and the like.

More specifically, for example, the following method can be exemplified.

A tubular base material (or frame) including annular support materials on the outer periphery is immersed in ethanol, and then the ethanol is completely removed by drying. The frame is subjected to a plasma treatment etc. A 1.0 to 3.0% by weight of collagen-containing dilute hydrochloric acid solution (0.001 N) (pH=about 3.0) is applied on the frame surface, and then air-dried. This application and air-drying operation is repeated to form a collagen coating on the frame surface. The coated frame is cooled to −20° C. preliminarily in a refrigerator.

A mold having a tubular gap capable of placing the tubular frame is prepared. The mold has an outer mold and a central rod-shaped body, and the side of the rod-shaped body serves as an inner mold. The frame coated with collagen is placed in the gap between the outer mold and the inner mold. Using a syringe, a 1-3% by weight of collagen-containing dilute hydrochloric acid (0.001 N) solution (pH=about 3.0) is loaded (or charged) between the outer mold and the inner mold. The collagen solution is frozen in a freezer. The frozen collagen solution is freeze-dried in a freeze-drying machine. A crosslinking treatment with heating is performed to obtain an artificial trachea.

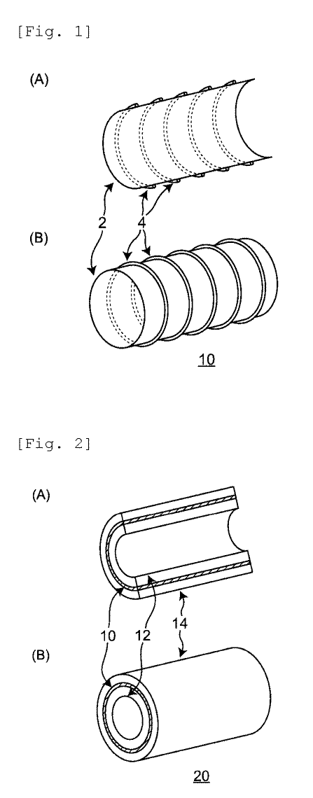

FIG. 1 schematically shows a frame (or backbone) (10) according to an embodiment of the present invention, comprising a tubular base material (2) having annular support materials (4) on the outer periphery of the tubular base material, the annular support materials (4) being placed apart from each other in an approximately equal interval on the outer periphery of the tubular base material (2). The tubular base material (2) and the annular support materials (4) are bonded to each other. FIG. 1(A) schematically displays the frame (10) viewed from the inside of the tubular base material (2) cut in the axial direction. FIG. 1 (B) schematically exhibits the whole frame (10).

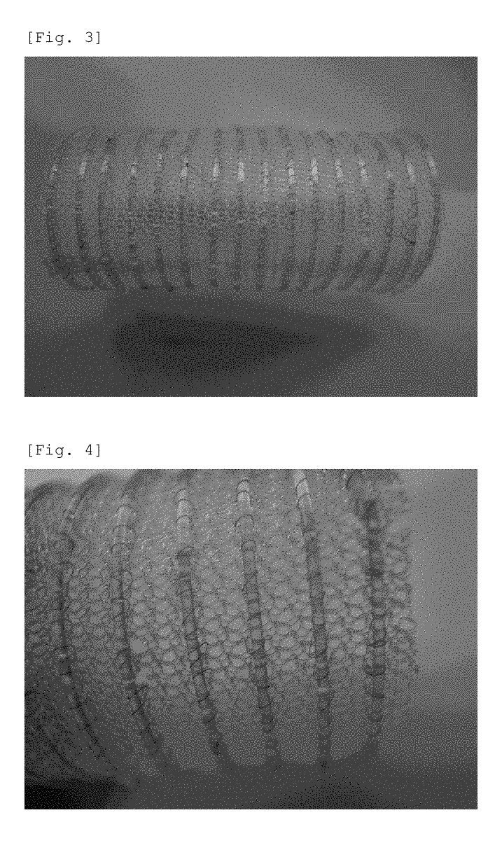

FIG. 2 schematically shows an artificial trachea (20) according to an embodiment of the present invention, having porous collagen layers (12, 14) on both the outside and the inside of the tubular frame (10) shown in FIG. 1. FIG. 2(A) schematically displays the artificial trachea (20) viewed from the inside of the artificial trachea (20) after being cut in the axial direction, and FIG. 2(B) schematically exhibits the whole artificial trachea (20).

The artificial trachea according to the embodiment of the present invention can be preferably used for reconstruction of a trachea and its tracheal bifurcation.

The artificial trachea according to the embodiment of the present invention has a simple and uncomplicated structure, and therefore has higher biocompatibility and is more easily incorporated into a living body.

Meanwhile, the artificial trachea according to the embodiment of the present invention is stable over a longer period of time inside the living body, and also has a mechanical strength capable of retaining the lumen of the artificial trachea over a longer period of time, and can maintain stable performance over a longer period of time, even though the artificial trachea has a simple and uncomplicated structure.

### EXAMPLES

The present invention will be described with specificity and detail below by way of Examples, but these Examples are merely embodiments of the present invention, and the present invention is not intended to be limited to these Examples in any way.

### Example 1

A polypropylene mesh (BIRD (registered trademark) mesh: BIRD mesh, manufactured by DAVOL INC.) was formed into a tubular shape to prepare a hollow tubular base material.

A polypropylene monofilament having a diameter of 0.994 mm (Monofilamente (trade name), manufactured by G. KRAHMER) was wound as a ring (or an annulus) around the tubular base material mentioned above to place an annular support material on the outer periphery of the tubular base material. The contact portion between the annular support material and the tubular base material was fused by melting with heating. Using a 7-0 prolene suture (polypropylene surgical suture Monofilamente (trade name), manufactured by G. KRAHMER), the annular support material was sewn (or fixed) on the tubular base material.

Similarly, annular support materials were placed on the annular base material at an interval of about 2 to 3 mm to obtain a frame (backbone) of Example 1 which comprised the annular support materials and the tubular base material having them on the outer periphery.

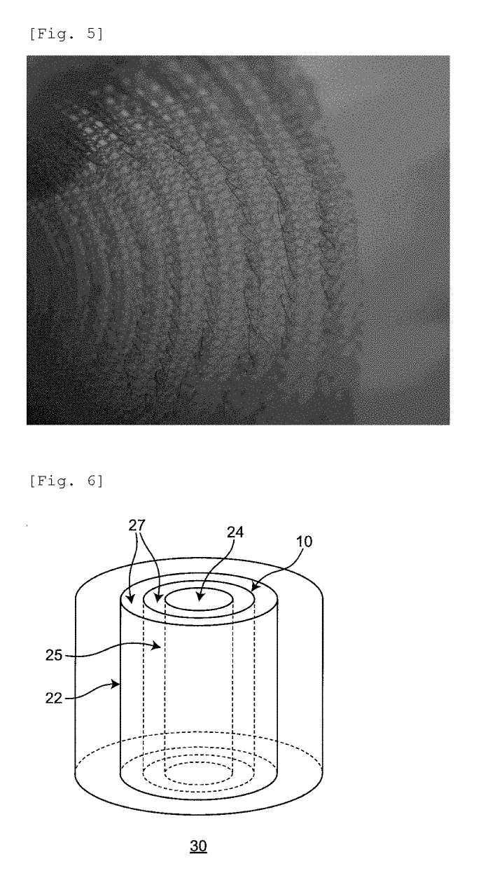

FIG. 3 shows a photograph of the frame of Example 1. FIG. 4 displays a photograph in which the outside of the side of the frame of Example 1 is enlarged. FIG. 5 exhibits a photograph in which the inside of the side of the frame of Example 1 is enlarged. These photographs apparently show that the annular support materials are placed on the outer periphery of the tubular base material.

The above-mentioned frame of Example 1 was immersed in 70% ethanol during a whole day and night (24 hours). The frame was taken out from the 70% ethanol, and then completely dried. The frame surface was treated with plasma. The outside of the frame was coated about twenty times with a 1-3% by weight of collagen (derived from dermis of 6-month-old swine (NMP collagen PSN (trade name)), manufactured by Nippon Meat Packers, Inc.)-containing dilute hydrochloric acid (0.001 N) solution (pH=about 3.0), and then dried.

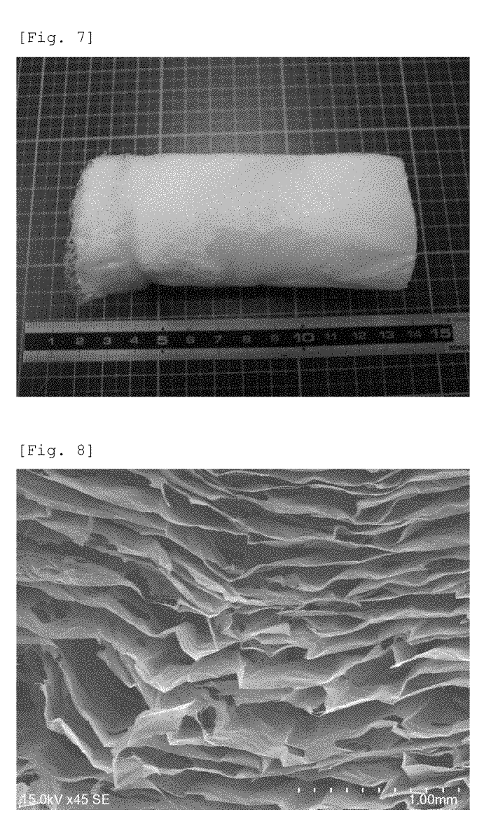

A mold with a tubular gap capable of placing the tubular frame is prepared. This mold is schematically shown in FIG. 6. The mold (30) has an outer mold (22) and a central rod-shaped body (24), and the side of the rod-shaped body serves as an inner mold (25). The frame coated with collagen was placed in a gap (27) between the outer mold (22) and the inner mold (25). Using a syringe, a 1-3% by weight of collagen (derived from dermis of swine (NMP collagen PSN (trade name)), manufactured by Nippon Meat Packers, Inc.)-containing dilute hydrochloric acid (0.001 N) solution (pH=about 3.0) was loaded (or charged) between the outer mold (22) and the inner mold (25). The collagen solution was frozen in a freezer. The frozen collagen solution was freeze-dried in a freeze-drying machine. A crosslinking treatment with heating was performed to obtain an artificial trachea of Example 1.

FIG. 7 shows a photograph of the artificial trachea of Example 1. The presence of a white outside porous collagen layer is well observed. The outer diameter of the outside collagen layer was about 4 cm, and the length thereof was about 12 cm. The artificial trachea of Example 1 absorbed blood well.

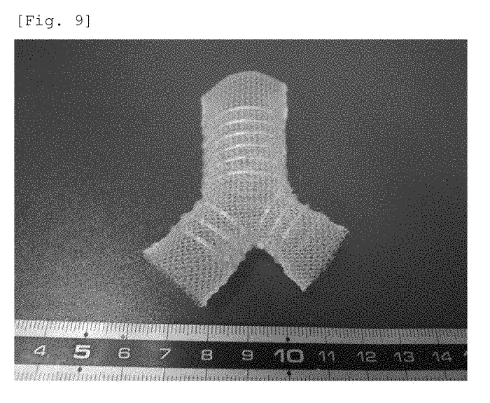

FIG. 8 displays a scanning electron micrograph (about 45 times magnification) of a cross-section of a collagen layer obtained by cutting the artificial trachea of Example 1 in a direction orthogonal to the axial direction. As a result, it has been recognized that the collagen layer has a porous structure. Pores having various sizes were observed, and the pores had various shapes. Therefore, the pores (shape and size) were non-uniform. However, the sizes of most pores are larger than those (about 5 to 20 μm) of general cells. Therefore, it has been found that the collagen layer has a structure which enables cells to invade into the collagen layer well. This collagen layer showed a thin film multilocular structure among collagens having a porous structure.

The artificial trachea of Example 1 was applied to a trachea of a beagle dog having a weight of 9 to 13 kg. After chest closure, it was confirmed that air leak of the artificial trachea was absent, and a chest tube (or drain) was immediately removed. After the surgery, an antibiotic was administered by intramuscular injection for a week, and then orally administered for 30 days. The beagle dog could survive for a long period of time with no complications.

### Example 2

In the same manner as in Example 1, except that a nylon monofilament having a diameter of 1.0 mm manufactured by Toray Industries, Inc. was wound as a ring around the tubular base material mentioned above to place annular support materials on the tubular base material, an artificial trachea of Example 2 was obtained.

### Example 3

A BIRD mesh was cut and sewn into a Y-shape to obtain a Y-shaped tubular base material. In the same manner as in Example 1, annular support materials were placed on each of the three Y-shaped branches to obtain a Y-shaped frame of Example 3. A Y-shaped mold was prepared, and collagen layers were placed on the frame of Example 3 using the same method as in Example 1 to obtain an artificial trachea of Example 3.

FIG. 9 exhibits a photograph of the frame of Example 3. The frame of Example 3 has branches and a Y-shaped form as a whole. Although each diameter of the branches of the tubular base material is slightly different, each annular support material can independently support each tubular base material firmly. Therefore, in the embodiment of the present invention, the tubular base material can be supported with an adequate strength without problems, even though the frame has a branch or even though the diameter of the tubular base material changes.

In the same manner as in Example 1, the artificial trachea of Example 3 was evaluated. The artificial trachea of Example 3 was applied to a trachea of a beagle dog having a weight of 9 to 13 kg. The beagle dog could survive for a long period of time with no complications.

An artificial trachea of the present invention has a simpler structure and is unlikely to be recognized as a foreign body in a living body, and the artificial trachea is more excellent in biocompatibility, and is more easily incorporated into a human body. After inserted into a living body, the artificial trachea is covered with a bronchial epithelium, and therefore can be completely integrated with the human body. Meanwhile, the artificial trachea according to the embodiment of the present invention has adequate mechanical strength capable of retaining the lumen over a longer period of time, and therefore can maintain stable performance over a longer period of time, even though the artificial trachea has a simpler structure.

### DESCRIPTION OF REFERENCE SYMBOLS

2: Tubular base material having meshes on the side
4: Annular support material(s)
10: Frame (backbone) comprising annular support materials and a tubular base material having the annular support materials on the outer periphery
12: Inside porous collagen layer
14: Outside porous collagen layer
20: Artificial trachea
22: Outer mold
24: Rod-shaped body
25: Inner mold
27: Gap
30: Mold

1. An artificial trachea comprising:
a hollow tubular base material whose cross-section in a direction orthogonal to the central axis of the tube has an approximately circular shape, the hollow tubular base material having meshes on the side of the tube;
a plurality of annular support materials that are placed apart from each other on the outer periphery of the tubular base material; and
porous collagen layers on both the outside and the inside of the tubular base material, wherein
the tubular base material is made of polyolefin,
the annular support material is made of at least one selected from polyolefin and polyamide, and
the tubular base material and the annular support materials are bonded to each other.

2. The artificial trachea according to claim 1, wherein the polyolefin comprises at least one selected from polypropylene, polyethylene and ethylene-propylene copolymer.

3. The artificial trachea according to claim 1, wherein the polyamide comprises at least one selected from 6,6-nylon and 6-nylon.

4. The artificial trachea according to claim 1, wherein the bonding of the tubular base material to the annular support materials is at least one selected from fusion bonding by heating and fixing with a suture.

5. The artificial trachea according to claim 4, wherein the suture comprises a suture made of at least one selected from polyolefin and polyamide.

6. The artificial trachea according to claim 1, wherein the porous collagen comprises at least one selected from sponge collagen, thin film multilocular collagen and fine fiber collagen.

7. The artificial trachea according to claim 1, wherein joint portions of three continuous annular support materials are placed so as not to be linearly aligned.

8. The artificial trachea according to claim 1, which has at least one form selected from a linear form, a curved form, a form having a constant diameter, a form having a non-constant diameter, a non-branching form, and a branching form.

9. A method for producing an artificial trachea, which comprises:
forming a mesh made of polyolefin in a hollow tubular shape to prepare a hollow tubular base material whose cross-section in a direction orthogonal to the central axis of the tube has an approximately circular shape, the hollow tubular base material having meshes on the side of the tube;
placing a plurality of annular support materials apart from each other on the outer periphery of the tubular base material using a monofilament made of at least one selected from polyolefin and polyamide;
bonding the annular support materials to the tubular base material; and
placing porous collagen layers on both the outside and the inside of the tubular base material.

10. The artificial trachea according to claim 2, wherein the polyamide comprises at least one selected from 6,6-nylon and 6-nylon.

11. The artificial trachea according to claim 2, wherein the bonding of the tubular base material to the annular support materials is at least one selected from fusion bonding by heating and fixing with a suture.

12. The artificial trachea according to claim 3, wherein the bonding of the tubular base material to the annular support materials is at least one selected from fusion bonding by heating and fixing with a suture.

13. The artificial trachea according to claim 2, wherein the porous collagen comprises at least one selected from sponge collagen, thin film multilocular collagen and fine fiber collagen.

14. The artificial trachea according to claim 3, wherein the porous collagen comprises at least one selected from sponge collagen, thin film multilocular collagen and fine fiber collagen.

15. The artificial trachea according to claim 4, wherein the porous collagen comprises at least one selected from sponge collagen, thin film multilocular collagen and fine fiber collagen.

16. The artificial trachea according to claim 5, wherein the porous collagen comprises at least one selected from sponge collagen, thin film multilocular collagen and fine fiber collagen.
